(19) United States
(12) Patent Application Publication
(10) Pub. No.: US 2024/0330771 A1
(43) Pub. Date: Oct. 3, 2024

(54) INFORMATION PROCESSING DEVICE, INFORMATION PROCESSING METHOD, PROGRAM RECORDING MEDIUM, AND MODEL BEING TRAINED

(71) Applicant: SEIKO EPSON CORPORATION, Tokyo (JP)

(72) Inventors: Yoshihiro YAMAMURA, Azumino-shi (JP); Yuya OZAWA, Azumino-shi (JP)

(73) Assignee: SEIKO EPSON CORPORATION, Tokyo (JP)

(21) Appl. No.: 18/618,679

(22) Filed: Mar. 27, 2024

(30) Foreign Application Priority Data
Mar. 28, 2023 (JP) 2023-050818

(51) Int. Cl. G06N 20/00

(52) U.S. Cl. CPC G06N 20/00

(57) ABSTRACT

Provided is an information processing device including a target person information acquisition unit configured to acquire target person information including age information indicating an age of a target person, an exercise evaluation information acquisition unit configured to acquire exercise evaluation information about evaluation of an exercise of the target person, a storage unit configured to store a trained model in which, when the target person information and the exercise evaluation information are input, evaluation of exercise information of the target person is estimated to be improved, and instruction content based on the age information is output, and a report output unit configured to input the target person information and the exercise evaluation information to the trained model and output an instruction report including the instruction content output from the trained model.

The present application is based on, and claims priority from JP Application Serial Number 2023-050818, filed Mar. 28, 2023, the disclosure of which is hereby incorporated by reference herein in its entirety.

### BACKGROUND

### 1. Technical Field

The present disclosure relates to an information processing device, an information processing method, a program recording medium, and a model being trained.

### 2. Related Art

In the walking instruction system described in JP-A-2021-74066, a walking index is determined in consideration of a subject' gender, age, height, weight, BMI, and disease, and a footprint image is displayed in accordance with the walking index (see JP-A-2021-74066).

However, in the known technique, since the footprint image is merely displayed, it is not possible to output training content or advice on training suitable for each target person, and in some cases, it is difficult to improve walking ability of the target person.

### SUMMARY

In order to solve the above problems, one aspect is an information processing device including a target person information acquisition unit configured to acquire target person information including age information indicating an age of a target person, an exercise evaluation information acquisition unit configured to acquire exercise evaluation information about evaluation of an exercise of the target person, a storage unit configured to store a trained model in which, when the target person information and the exercise evaluation information are input, evaluation of exercise information of the target person is estimated to be improved, and instruction content based on the age information is output, and a report output unit configured to input the target person information and the exercise evaluation information to the trained model and output an instruction report including the instruction content output from the trained model.

In order to solve the above problems, one aspect is an information processing method including acquiring target person information including age information indicating an age of a target person using a target person information acquisition unit of an information processing device, acquiring exercise evaluation information about evaluation of an exercise of the target person using an exercise evaluation information acquisition unit of the information processing device, storing, using a storage unit of the information processing device, a trained model in which, when the target person information and the exercise evaluation information are input, evaluation of exercise information of the target person is estimated to be improved, and instruction content based on the age information is output, and inputting the target person information and the exercise evaluation information to the trained model and outputting an instruction report including the instruction content output from the trained model using a report output unit of the information processing device.

In order to solve the above problems, one aspect is a program recording medium configured to record a program, the program causing a computer to execute a target person information acquisition function configured to acquire target person information including age information indicating an age of a target person, an exercise evaluation information acquisition function configured to acquire exercise evaluation information about evaluation of an exercise of the target person, and a report output function configured to input the target person information and the exercise evaluation information to a trained model stored in a storage unit configured to store the trained model in which, when the target person information and the exercise evaluation information are input, evaluation of exercise information of the target person is estimated to be improved, and instruction content based on the age information is output, and output an instruction report including the instruction content output from the trained model.

In order to solve the above problems, one aspect is an information processing device including an instruction content acquisition unit configured to acquire instruction content based on target person information including a plurality of parameters related to a target person and exercise evaluation information about evaluation of an exercise of the target person, an exercise information evaluation unit configured to acquire the exercise evaluation information about the evaluation of the exercise of the target person after the instruction, and a storage unit configured to store a model being trained in which, when the instruction content and the exercise evaluation information after the instruction are input, an effective parameter among the plurality of parameters of the target person information is analyzed.

In order to solve the problems, one aspect is a model being trained in which, when instruction content based on target person information including a plurality of parameters related to a target person and exercise evaluation information about evaluation of an exercise of the target person, and the exercise evaluation information after the instruction are input, an effective parameter among the plurality of parameters of the target person information is analyzed.

In order to solve the above problems, one aspect is an information processing method including acquiring instruction content based on target person information including a plurality of parameters related to a target person and exercise evaluation information about evaluation of an exercise of the target person using an instruction content acquisition unit of an information processing device, acquiring the exercise evaluation information about the evaluation of the exercise of the target person after the instruction using an exercise information evaluation unit of the information processing device, and storing, using a storage unit of the information processing device, a model being trained in which, when the instruction content and the exercise evaluation information after the instruction are input, an effective parameter among the plurality of parameters of the target person information is analyzed.

### BRIEF DESCRIPTION OF THE DRAWINGS

FIG. 1 is a diagram showing a schematic configuration example of an information processing device according to an embodiment.

FIG. 2 is a diagram showing an example of input and output of a trained model according to the embodiment.

FIG. 3 is a diagram showing a first table representing examples of categories, stages, and states of self-standing, frailty, support-required, and care-required according to the embodiment.

FIG. 4 is a diagram showing a second table representing J-CHS criteria according to the embodiment.

FIG. 5 is a diagram showing a third table representing personal information according to the embodiment.

FIG. 6 is a diagram showing a fourth table representing an example of determination criteria of obesity according to the embodiment.

FIG. 7 is a diagram showing a fifth table representing another example of determination criteria of obesity according to the embodiment.

FIG. 8 is a diagram showing an example of questionnaire content according to the embodiment.

FIG. 9 is a diagram showing a sixth table representing an example of classification of a personality according to the embodiment.

FIG. 10 is a diagram showing a seventh table representing personal information when the target person information according to the embodiment is the first pattern.

FIG. 11 is a diagram showing an eighth table representing personal information when the target person information according to the embodiment is a second pattern.

FIG. 12 is a diagram showing a ninth table representing personal information when the target person information according to the embodiment is a third pattern.

FIG. 13 is a diagram showing an example of waveform data of walking of a target person according to the embodiment.

FIG. 14 is a diagram showing another example of the waveform data of the walking of the target person according to the embodiment.

FIG. 15 is a diagram showing an example of a foot angle of the target person according to the embodiment.

FIG. 16 is a diagram showing an example of a movement of a lower half body of the target person according to the embodiment.

FIG. 17 is a diagram showing an example of a movement of an upper half body of the target person according to the embodiment.

FIG. 18 is a diagram showing a tenth table representing an example of ideal stride lengths by age according to the embodiment.

FIG. 19 is a diagram showing an eleventh table representing an example of ideal walking speed by age according to the embodiment.

FIG. 20 is a diagram showing an example of a first walking characteristic in a first pattern according to the embodiment.

FIG. 21 is a diagram showing a twelfth table representing an example of evaluation information in the first pattern according to the embodiment.

FIG. 22 is a diagram showing an example of a second walking characteristic in a second pattern according to the embodiment.

FIG. 23 is a diagram showing a thirteenth table representing an example of evaluation information in the second pattern according to the embodiment.

FIG. 24 is a diagram showing an example of a third walking characteristic in a third pattern according to the embodiment.

FIG. 25 is a diagram showing a fourteenth table representing an example of evaluation information in the third pattern according to the embodiment.

FIG. 26 is a diagram showing a fifteenth table representing an example of training classification according to the embodiment.

FIG. 27 is a diagram schematically showing an example of generation of teacher data according to the embodiment.

FIG. 28 is a diagram schematically showing an example of analysis using the learning model according to the embodiment.

FIG. 29 is a diagram schematically showing an example of analysis content in the learning model according to the embodiment.

### DESCRIPTION OF EMBODIMENTS

Embodiments will be described with reference to the drawings.

An embodiment will be described.

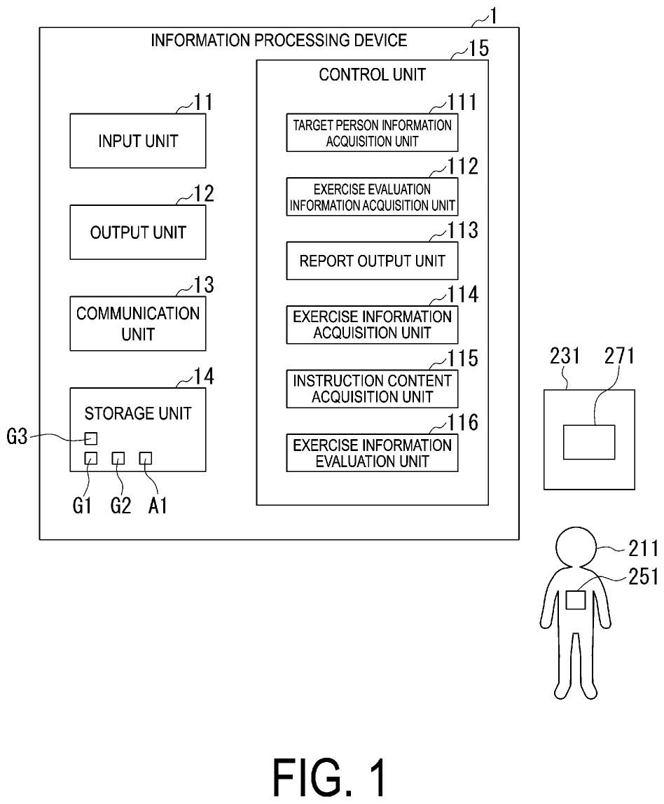

FIG. 1 is a diagram showing a schematic configuration example of an information processing device 1 according to the embodiment.

The information processing device 1 includes an input unit 11, an output unit 12, a communication unit 13, a storage unit 14, and a control unit 15.

The control unit 15 includes a target person information acquisition unit 111, an exercise evaluation information acquisition unit 112, a report output unit 113, an exercise information acquisition unit 114, an instruction content acquisition unit 115, and an exercise information evaluation unit 116.

Also, FIG. 1 shows a target person 211 and a first detection device 251 attached to the target person 211.

The target person 211 is a person who receives instruction of training.

Although FIG. 1 shows one target person 211, a plurality of target persons are present in this embodiment.

The first detection device 251 is a device that detects information for evaluating an exercise performed by the target person 211. The information may be information of any physical quantity and may be, for example, acceleration, position information, angular velocity, or the like.

Here, as another example, a tool 231 and a second detection device 271 attached to the tool 231 may be used.

The tool 231 is a tool used in training performed by the target person 211.

The second detection device 271 is a device that detects information for evaluating an exercise performed by the target person 211. The information may be information of any physical quantity and may be, for example, acceleration, position information, or angular velocity.

Further, for example, any one of the first detection device 251 and the second detection device 271 may be used, or both may be used.

Also, each of the first detection device 251 and the second detection device 271 may be configured using, for example, sensors that detect desired information.

The information detected by the first detection device 251 and the second detection device 271 may be called data, detected values, or the like.

The input unit 11 performs, for example, processing of inputting information in accordance with a user's operation and processing of inputting information from another device.

The output unit 12 performs, for example, processing of outputting information to another device. The other device may be a display device, and in this case, the information output from the output unit 12 is displayed on a screen of the display device. As another example, the other device may be a printing device, and in this case, the information output from the output unit 12 is printed out by the printing device.

The communication unit 13 communicates with other devices. The communication unit 13 is configured to communicate with, for example, the first detection device 251 and the second detection device 271. In addition, the communication unit 13 may be configured to communicate with other devices (not shown) such as a printing device.

Also, in this embodiment, the communication unit 13 is shown separately from the input unit 11 and the output unit 12, but the function of the communication unit 13 may be included in the functions of the input unit 11 and the output unit 12, for example.

The storage unit 14 stores information.

In this embodiment, the storage unit 14 stores a learning model A1, target person information G1, exercise evaluation information G2, and exercise information G3.

The learning model A1 is a model for machine learning and may be, for example, a pre-learning state, an on-learning state, and a learned state.

The target person information G1 is information about the target person 211.

The exercise evaluation information G2 is information indicating evaluation of an exercise of the target person 211.

The exercise information G3 is information about the exercise of the target person 211.

Also, two or more of the target person information G1, the exercise evaluation information G2, and the exercise information G3 may be associated with each target person 211. The association may be called correspondence or the like.

Here, for example, the information processing device 1 may acquire the exercise evaluation information G2 of the target person 211 from the outside, or may acquire the exercise information G3 of the target person 211 from the outside and generate the exercise evaluation information G2 based on the acquired exercise information G3.

Also, in the example shown in FIG. 1, a state in which the learning model A1, the target person information G1, the exercise evaluation information G2, and the exercise information G3 are stored in the storage unit 14 is shown, but there may be a state in which some or all of these are not stored in the storage unit 14 depending on a timing of processing or the like.

The control unit 15 performs various types of processing and control.

In this embodiment, the information processing device 1 is configured using a computer.

The control unit 15 includes a processor such as a central processing unit (CPU) and performs various types of processing and control by executing a predetermined program using the processor.

The program may be stored in the storage unit 14, for example.

The target person information acquisition unit 111 acquires the target person information G1 via, for example, the input unit 11, the communication unit 13, or the storage unit 14.

The exercise evaluation information acquisition unit 112 acquires the exercise evaluation information G2 via, for example, the input unit 11, the communication unit 13, or the storage unit 14.

The report output unit 113 generates an instruction report H1 and outputs the instruction report H1. This output may be, for example, a display output or a paper output from a printing device.

Here, the instruction report H1 includes instruction content.

In addition, the instruction report H1 may include, for the target person 211, for example, exercise evaluation information before the instruction report H1 was issued in the past, may include exercise evaluation information after the instruction report H1 was issued in the past, or may include both of these.

The exercise information acquisition unit 114 acquires the exercise information G3 via, for example, the input unit 11, the communication unit 13, or the storage unit 14.

Here, for example, the exercise evaluation information acquisition unit 112 may generate and acquire the exercise evaluation information G2 based on the exercise information G3 acquired by the exercise information acquisition unit 114 and a predetermined evaluation rule.

The evaluation rule is a rule for evaluating the exercise information G3 and may include ideal exercise information, for example.

The instruction content acquisition unit 115 acquires information about the instruction content transmitted to the target person 211.

The exercise information evaluation unit 116 acquires exercise evaluation information about the target person 211 to whom the instruction content is transmitted.

Here, for example, the exercise information evaluation unit 116 may acquire such exercise evaluation information via the input unit 11, the communication unit 13, or the storage unit 14, or may acquire exercise information of the target person 211 via the input unit 11, the communication unit 13, or the storage unit 14 and acquire exercise evaluation information based on the exercise information.

In this embodiment, the instruction content acquisition unit 115 acquires content of instruction based on the target person information G1 including a plurality of parameters related to the target person 211 and the exercise evaluation information G2 about the evaluation of the exercise of the target person 211.

In addition, the exercise information evaluation unit 116 acquires the exercise evaluation information about the evaluation of the exercise of the target person 211 after the instruction.

When the content of instruction and the exercise evaluation information after the instruction are input, the learning model A1 during learning analyzes effective parameters among the plurality of parameters of the target person information G1.

Input and output of a trained model A2 will be described.

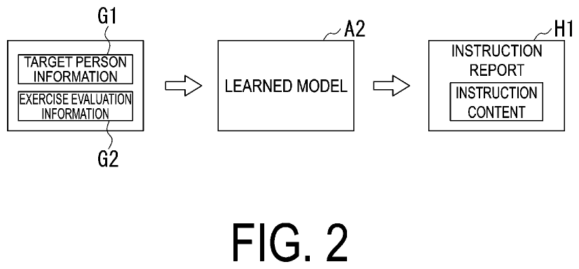

FIG. 2 is a diagram showing an example of input and output of the trained model A2 according to the embodiment.

The trained model A2 is a model in which the learning model A1 is in a learned state.

In this embodiment, the target person information G1 and the exercise evaluation information G2 are input to the trained model A2, and based on instruction content output from the trained model A2, the instruction report H1 including the instruction content is output.

Here, the trained model A2 does not necessarily need to output sentences, patterns, or the like of the instruction content as they are, and may output, for example, information that can identify the instruction content. As an example, a correspondence relationship between each of a plurality of pieces of identification information including numbers or the like and instruction content may be stored in a database or the like, the trained model A2 may output one or more pieces of identification information, and the instruction content corresponding to the identification information may be identified and included in the instruction report H1.

The target person 211 will be described.

In this embodiment, an elderly person of 65 years or older who is classified as frail is set as the target person 211, and based on the target person information G1 which is personal information of the target person 211 and the exercise evaluation information G2 which is evaluation of walking acquired from the target person 211, the instruction report H1 describing personalized rehabilitation training and advice is presented, thereby reducing the risk of the target person 211 being in a support-required or care-required state.

Here, in this embodiment, the target person 211 is an elderly person of 65 years or older, but is not limited thereto.

Also, in this embodiment, frailty also includes a pre-frail group.

The personal information is, for example, information such as age, gender, living environment, and the like.

In this embodiment, the target person 211 is an elderly person serving as a target.

The support-required and the care-required will be described.

Steps of the support-required and the care-required are described.

Support-required 1 is a state in which basic activities of daily life can be performed by oneself, but supervision or assistance is required for some activities. The time required for such care is 25 to 31 minutes/day.

Support-required 2 is a state in which muscle strength is weakened and there is instability in walking and standing up, and there is a high likelihood that care will be required. The time required for such care is 32 to 49 minutes/day.

Care-required 1 is a state in which some assistance is required for daily life, standing up, and walking, and a slight cognitive decline is observed. The time required for the care is 32 to 49 minutes/day.

Care-required 2 is a state in which care is required for activities of daily life more than in Care-required 1 and a cognitive decline is observed. The time required for the care is 50 to 69 minutes/day.

Care-required 3 is a state in which general assistance is required for activities of daily life and a cane, a walker, or a wheelchair is used for standing up or walking, and cognitive functions have declined, and supervision is also required. The time required for the care is 70 to 89 minutes/day.

Care-required 4 is a state in which assistance is required in all aspects of life more than in Care-required 3, and significant decreases in thinking ability and understanding ability are observed. The time required for the care is 90 to 109 minutes/day.

Care-required 5 is a state in which assistance is required throughout daily life and communication is also difficult. The time required for the care is 110 minutes/day or more.

Frailty will be described.

Frailty indicates a state between a self-standing state and a support-required state.

It is difficult for support-required and care-required target persons to recover to the extent that they can stand for themselves even if they perform training, whereas frail target persons can recover by performing training to the extent that they can stand for themselves.

There are various determination criteria of frailty, and J-CHS criteria based on cardiovascular health study (CHS) criteria are adopted in Japan.

The J-CHS criteria are determination criteria of frailty when three or more of five criteria items are satisfied.

One of the J-CHS criteria relates to a walking speed, and in this embodiment, improving the walking speed promotes self-standing of the frail and reduces the risk of becoming support-required and care-required.

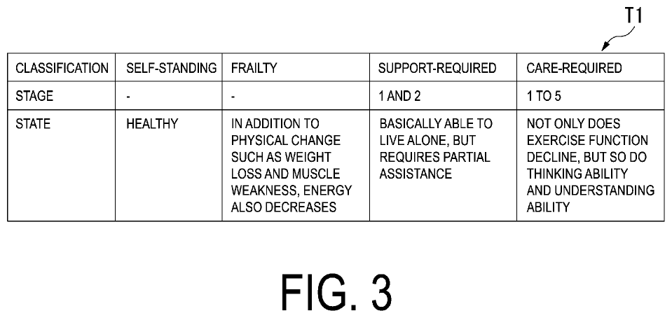

FIG. 3 is a diagram showing a first table T1 representing an example of classifications, stages, and states of self-standing, frailty, support-required, and care-required according to the embodiment.

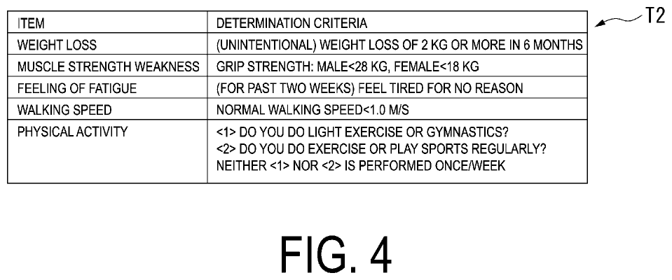

FIG. 4 is a diagram showing a second table T2 representing the J-CHS criteria according to the embodiment.

Personal information, which is an example of the target person information G1, will be described.

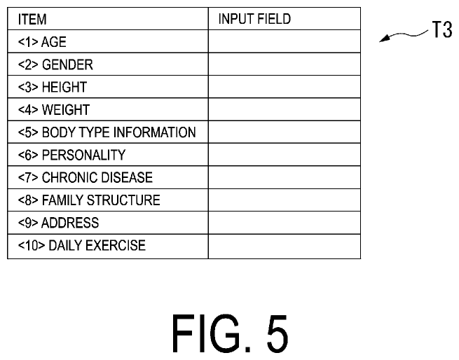

FIG. 5 is a diagram showing a third table T3 representing the personal information according to the embodiment.

The personal information of the target person will be described.

In this embodiment, the personal information is used as the target person information G1 which is input information for machine learning.

The personal information includes, for example, information about items such as <1> an age, <2> a gender, <3> a height, <4> a weight, <5> a body type information, <6> a personality, <7> a chronic disease, <8> a family structure, <9> an address, and <10> a daily exercise.

The personal information is input via a predetermined information processing device, for example.

For example, the personal information of the target person 211 may be input by the target person 211, or may be input by an instructor who gives training instruction to the target person 211, or a person concerned with the target person 211. The concerned person is, for example, a family member of the target person 211.

Here, the information processing device may be, for example, a personal computer, a smartphone, an electronic medical record, or the like.

Also, for example, the information processing device 1 according to this embodiment may be used for the information processing device, but when another information processing device is used, the personal information is transmitted from the other information processing device to the information processing device according to this embodiment.

<1> The age is information indicating an age of the target person 211.

<2> The gender is information indicating a gender of the target person 211.

<3> The height is information indicating a height of the target person 211.

<4> The weight is information indicating a weight of the target person 211.

<5> The body type information is information about a body type of the target person 211.

Here, for example, the body type information may be input by the target person 211, may be input as a body mass index (BMI) derived from the height and the weight of the target person, or may be input by determination based on the BMI.

BMI is a body mass index representing a degree of obesity calculated from a weight and a height and is obtained by weight [kg]/height [m]².

In this embodiment, a case of using determination criteria of the Japan Society for the Study of Obesity is shown as an example, but as another example, determination criteria of the World Health Organization (WHO) may be used.

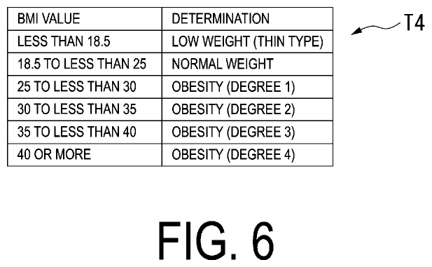

FIG. 6 is a diagram showing a fourth table T4 representing an example of determination criteria of obesity according to the embodiment.

In the fourth table T4, the determination criteria of the Japan Society for the Study of Obesity are shown.

In the determination criteria, correspondence between BMI values and determinations is defined.

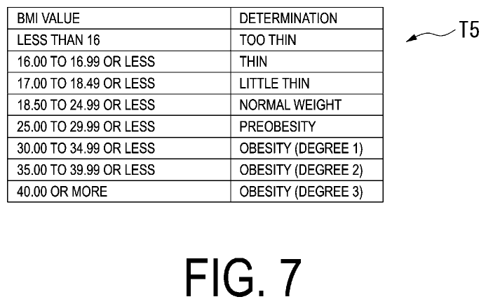

FIG. 7 is a diagram showing a fifth table T5 representing another example of the determination criteria of obesity according to the embodiment.

In the fifth table T5, the determination criteria of the World Health Organization (WHO) are shown.

In the determination criteria, correspondence between BMI values and determinations is defined.

<6> The personality is information about a personality of the target person 211.

Here, the information about the personality of the target person 211 is, for example, a classification of the personality derived from the results obtained from answers of the target person 211 to a questionnaire.

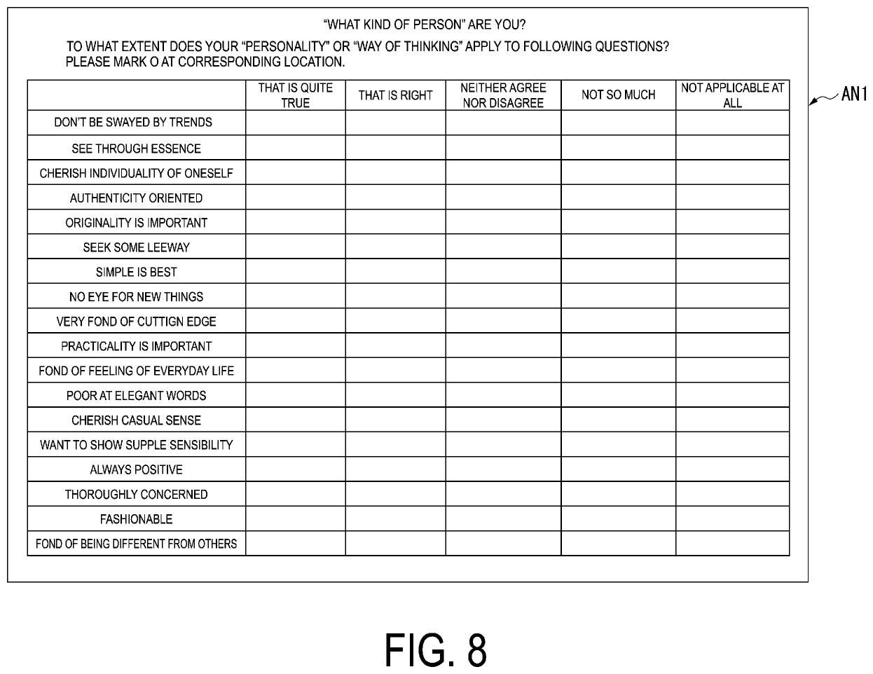

FIG. 8 is a diagram showing an example of questionnaire content AN1 according to the embodiment (see JP-A-2004-164087).

Here, any content may be used as the questionnaire content. For example, the questionnaire content may be selective content or may be descriptive content.

Also, the questionnaire may also be called a question, inquiry, or the like.

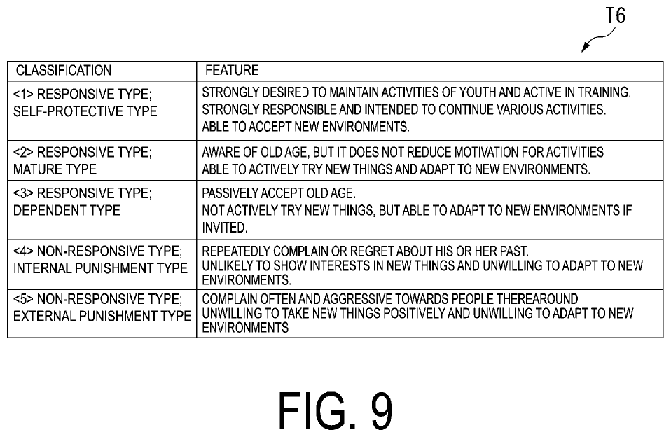

FIG. 9 is a diagram showing a sixth table T6 representing an example of classification of the personality according to the embodiment.

In the sixth table T6, correspondence between classification of the personality and features is defined.

In this embodiment, the personality is classified into five types, that is, <1> a responsive type; a self-protective type, <2> a responsive type; a mature type, <3> a responsive type; a dependent type, <4> a non-responsive type; an internal punishment type, and <5> a non-responsive type; an external punishment type.

As a reference to this, there are five personality types in old age by Suzanne Reichard.

Here, in this embodiment, since the target person is an elderly person among those classified as being frail, classification of a personality of an elderly person is used, but the present disclosure is not limited thereto, and as long as a subject is classified as being frail, any subject whose personality can be classified may be used.

Further, in this embodiment, a personality of the target person is classified, but the present disclosure is not limited thereto, and the results of the questionnaire may be used without classifying the personality.

Also, classification of the personality is not limited to the example of this embodiment, and another classification may be used, and for example, classification based on a level of motivation for training, classification based on a strength of intention to continue training, classification based on a strength of oneself, such as whether or not one can accept advice frankly, or the like may also be used.

<7> The chronic disease is information about a chronic disease of the target person 211.

The chronic disease includes, for example, back pain, knee pain, five major lifestyle-related diseases, and the like.

<8> The family structure is information about cohabitants.

The information about cohabitants also includes, for example, information about the number of cohabitants. and attributes of the cohabitants. The attributes include, for example, a wife, a son, a daughter-in-law, and the like.

<9> The address is information about an environment in which the target person 211 lives.

From the address, it is possible to determine whether or not the target person 211 lives in an urban area, or whether or not the target person 211 lives in a mountainous area or the like.

<10> The daily exercise is information about an exercise performed by the target person 211 on a daily basis.

The daily exercise includes, for example, farm work, walking, gymnastics, gate ball, and the like.

A specific example of the personal information will be described.

In this embodiment, as the specific example, personal information when the target person information G1 is a first pattern, a second pattern, and a third pattern will be described.

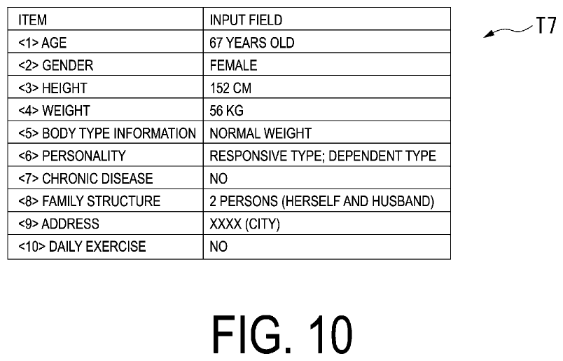

FIG. 10 is a diagram showing a seventh table T7 representing personal information when the target person information G1 according to the embodiment is the first pattern.

In the first pattern, <1> the age is 67 years old, <2> the gender is female, <3> the height is 152 cm, <4> the weight is 56 kg, <5> the body type information is a normal weight, <6> the personality is a responsive type; a dependent type, <7> no chronic disease, <8> the family structure is two persons, that is, herself and her husband, <9> the address is “xxxx,” which is a city, and <10> no daily exercise.

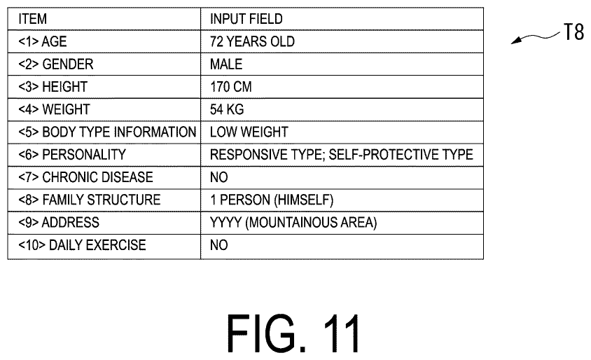

FIG. 11 is a diagram showing an eighth table T8 representing personal information when the target person information G1 according to the embodiment is the second pattern.

In the second pattern, <1> the age is 72 years old, <2> the gender is male, <3> the height is 170 cm, <4> the weight is 54 kg, <5> the body type information is low weight, <6> the personality is a responsive type; a self-protective type, <7> no chronic disease, <8> the family structure is one person, that is, himself, <9> the address is “yyyy,” which is a mountainous area, and <10> no daily exercise.

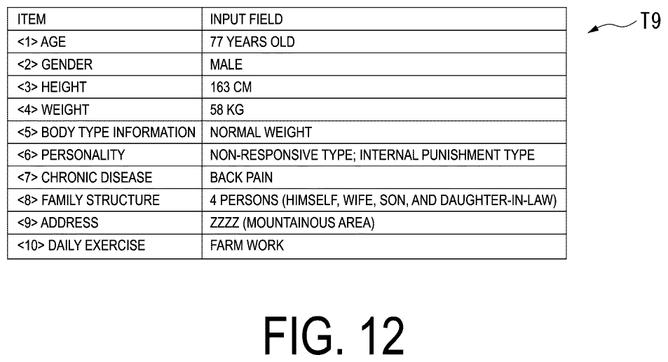

FIG. 12 is a diagram showing a ninth table T9 representing personal information when the target person information G1 according to the embodiment is the third pattern.

In the third pattern, <1> the age is 77 years old, <2> the gender is male, <3> the height is 163 cm, <4> the weight is 58 kg, <5> the body type information is a normal weight, <6> the personality is a non-responsive type; an internal punishment type, <7> the chronic disease is back pain, <8> the family constitution is four persons, that is, himself, his wife, son, and daughter-in-law, <9> the address is “zzzz,” which is a mountainous area, and <10> no daily exercise.

Information about walking of the target person 211, which is an example of the exercise evaluation information, will be described.

Information including evaluation of walking acquired from the target person will be described.

In this embodiment, the information is used as the exercise evaluation information G2 which is input information of machine learning.

The information about the walking of the target person 211 is acquired by a sensor of the first detection device 251 attached to the target person 211.

In this embodiment, the sensor is a device that acquires acceleration and angular velocity and is attached to a predetermined portion of the body of the target person 211. The predetermined portion may be, for example, a foot, a waist, or the like. The present disclosure is not limited thereto, and the sensor may be attached to a leg, an arm, a torso, or a neck.

The information about the walking includes, for example, <1> a walking style, <2> a foot angle, <3> a movement of a lower half body, <4> a movement of an upper half body, and the like.

### <1> Walking Style

The walking style includes, for example, information about a stride length, a walking speed, and a standing time on both feet.

The stride length is determined by acquiring waveform data for each of left and right feet using sensors attached to the left and right feet of the target person 211 and calculating a distance of the stride length from the waveform data.

Here, since the waveform data indicates a peak value when a foot is in contact with the ground, a cycle between peak values can be defined as one walking cycle. For example, one walking cycle of the right foot may be used, one walking cycle of the left foot may be used, or an average value of one walking cycle of the right foot and one walking cycle of the left foot may be used.

The stride length of the target person 211 can be calculated from a distance traveled by the target person 211 and the number of walking cycles.

In addition, since an amplitude of the acceleration increases in proportion to a speed at which a foot is raised or lowered, if the amplitude is small, the speed at which the foot is raised or lowered is slow, and thus the stride length is likely to be shorter.

The walking speed can be calculated from a walking cycle per unit time.

The standing time on both feet can be calculated from the time during which a peak value of the right foot and a peak value of the left foot overlap each other in the waveform data.

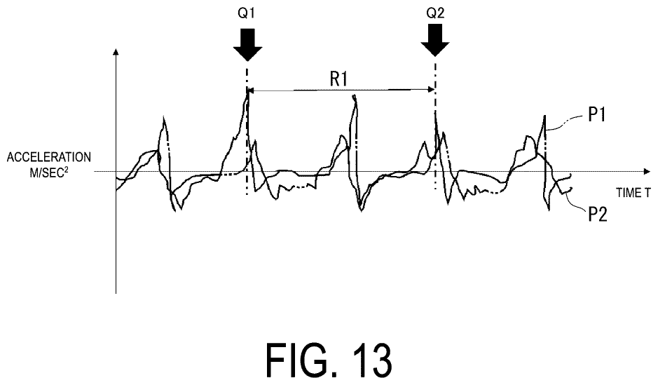

FIG. 13 is a diagram showing an example of the waveform data of the walking of the target person 211 according to the embodiment.

In the graph shown in FIG. 13, the horizontal axis represents time [t] and the vertical axis represents acceleration [m/sec²].

The graph shows a first right foot characteristic P1 of the right foot of the target person 211 and a first left foot characteristic P2 of the left foot of the target person 211.

In addition, FIG. 13 shows a first ground contact timing Q1 and a second ground contact timing Q2 indicating that the left foot is in contact with the ground, and a first walking period R1 which is one walking period corresponding to a time required for a first walking cycle.

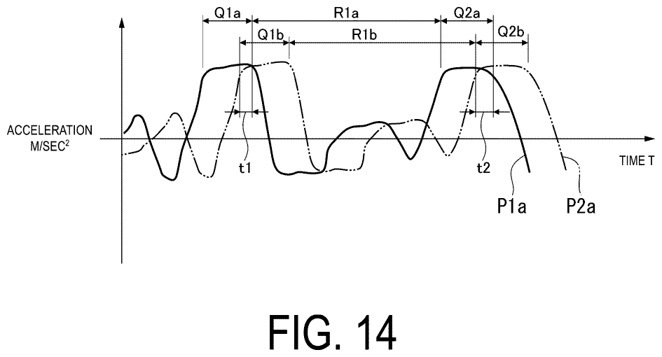

FIG. 14 is a diagram showing another example of the waveform data of the walking of the target person 211 according to the embodiment.

In the graph shown in FIG. 14, the horizontal axis represents time [t] and the vertical axis represents acceleration [m/sec²].

The graph shows a 1a right foot characteristic Pla of the right foot of the target person 211 and a 1a left foot characteristic P2a of the left foot of the target person 211.

In addition, FIG. 14 shows a 1a ground contact timing Q1a and a 2a ground contact timing Q2a indicating that the right foot is in contact with the ground, a 1a walking period R1a which is one walking period corresponding to the time required for the first walking cycle, a 1b ground contact timing Q1b and a 2b ground contact timing Q2b indicating that the left foot is in contact with the ground, a 1b walking period Rib which is one walking period corresponding to the time required for the first walking cycle, a first overlap time t1 indicating a time during which the 1a ground contact timing Q1a overlaps the 1b ground contact timing Q1b, and a second overlap time t2 indicating a time during which the 2a ground contact timing Q2a overlaps the 2b ground contact timing Q2b.

### <2> Foot Angle

The foot angle includes, for example, information about a foot opening angle and a toe angle.

The foot opening angle is information indicating an angle formed by the left foot and the right foot. When an angle related to the foot opening angle is large, the angle formed by the entire right leg and the entire left leg becomes large, which places a load to the pelvis.

The toe angle is information indicating an angle formed between the ground and the toe. When the toe angle is small, there is a risk of falling when the toe hits the ground.

In this embodiment, the foot angle is calculated by a sensor attached to a foot of the target person 211. The sensor is, for example, a sensor that detects angular velocity.

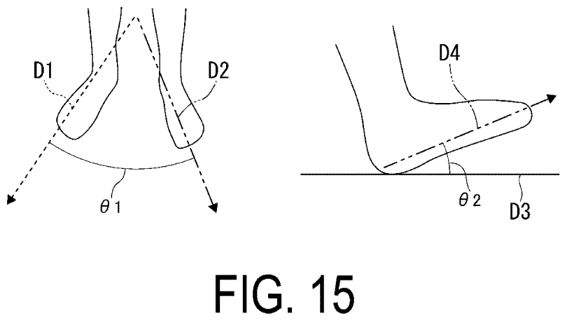

FIG. 15 is a diagram showing an example of the foot angle of the target person 211 according to the embodiment.

FIG. 15 shows a first right foot direction D1 which is a direction of the right foot of the target person 211, a first left foot direction D2 which is a direction of the left foot of the target person 211, and a first angle θ1 which is an opening angle between the first right foot direction D1 and the first left foot direction D2.

In addition, FIG. 15 shows, for one foot of the target person 211, a first ground direction D3 which is a direction of the ground, a first toe direction D4 which is a direction of toes of the foot, and a second angle θ2 which is an angle between the first ground direction D3 and the first toe direction D4.

### <3> Movement of Lower Half Body

The movement of the lower half body includes, for example, information about a flexion angle of a knee joint and a leg opening angle of a hip joint.

The flexion angle of the knee joint is information indicating an angle formed by a direction upward from the knee to the thigh and a direction downward from the knee to the calf.

The leg opening angle of the hip joint is information indicating an angle formed by the right leg and the left leg.

When the flexion angle of the knee joint and the leg opening angle of the hip joint are small, it becomes impossible to move the foot far forward, and the stride length becomes shorter.

In this embodiment, the movement of the lower half body is calculated by sensors attached to the waist and the feet. The sensor is a sensor that detects angular velocity.

Further, by attaching a sensor to the knee as well, it is possible to increase information acquisition accuracy.

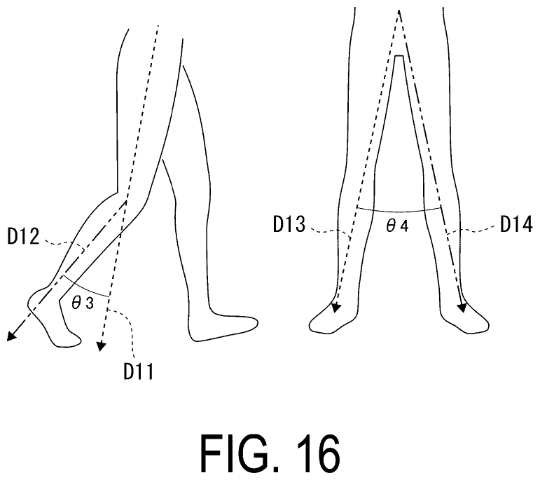

FIG. 16 is a diagram showing an example of the movement of the lower half body of the target person 211 according to the embodiment.

FIG. 16 shows a first knee upward direction D11 of the target person 211, a first knee downward direction D12 of the target person 211, and a third angle θ3 which is a flexion angle of the knee joint corresponding to an angle between the first knee upward direction D11 and the first knee downward direction D12.

In addition, FIG. 16 shows a first right leg direction D13 which is a direction of the right leg of the target person 211, a first left leg direction D14 which is a direction of the left leg of the target person 211, and a fourth angle θ4 which is a leg opening angle of the hip joint corresponding to an angle between the first right leg direction D13 and the first left leg direction D14.

### <4> Movement of Upper Half Body

The movement of the upper half body includes, for example, information about a vertical movement, a horizontal movement, and a forward tilt of the upper body.

The vertical movement of the upper body is information indicating a vertical movement of the upper half body during walking. When the vertical movement of the upper body is small, it is impossible to move the foot far forward, and thus the stride length becomes shorter.

The horizontal movement of the upper body is information indicating rightward and leftward sways of the upper half body during walking. When the horizontal movement of the upper body is large, loads applied to the waist and the knee are increased, and there is a risk of injury.

The forward tilt indicates an angle at which the upper half body is inclined forward. When the forward tilt is large, the walking speed decreases.

In this embodiment, the movement of the upper half body is calculated by a sensor attached to the waist of the target person 211. The sensor is, for example, a sensor that detects one or both of acceleration and angular velocity.

As another example, one or both of information about the arm and information about the neck may be acquired by attaching a sensor to the arm or the neck of the target person 211. The information about the arms includes, for example, information about bending of an elbow joint or a range of movement of an upper limb. The information about the neck includes, for example, information about a direction of a line of sight.

By acquiring the information about the arm and the information about the neck, it is possible to acquire the information about the walking of the target person 211 with high accuracy.

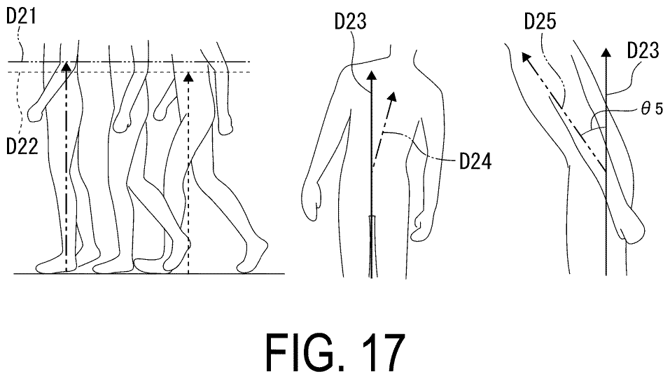

FIG. 17 is a diagram showing an example of the movement of the upper half body of the target person 211 according to the embodiment.

FIG. 17 shows, in the vertical movement of the upper body of the target person 211, a first upper position D21 which is an upper position of the upper body, and a first lower position D22 which is a lower position of the upper body.

Also, FIG. 17 shows, in the vertical movement of the upper body of the target person 211, a first upward direction D23 which is an upward direction with respect to the ground, and a first upper half body direction D24 which is a direction of the upper half body of the upper body.

In addition, FIG. 17 shows, in the forward tilt of the upper body, a first upward direction D23 which is an upward direction with respect to the ground, a first forward direction D25 which is a forward direction of the upper body, and a fifth angle θ5 which is a forward tilt of the upper body corresponding to an angle between the first upward direction D23 and the first forward direction D25.

Information about evaluation of the walking of the target person 211, which is an example of the exercise evaluation information G2, will be described.

The evaluation according to this embodiment is evaluation of the walking of the target person compared with walking of ideal data.

The evaluation is, for example, comparison in numerical data, comparison in waveform data, a comment based on the comparison in waveform data, or the like.

In this embodiment, content of the evaluation is a comment based on comparison in waveform data.

For the evaluation, for example, evaluation of each of <1> the walking style, <2> the foot angle, <3> the movement of the lower half body, and <4> the movement of the upper half body, which are information about the walking, may be used, or evaluation in which all of them are summarized may be used.

In this embodiment, evaluation is performed for each of <1> the walking style, <2> the foot angle, <3> the movement of the lower half body, and <4> the movement of the upper half body, which are information about the walking.

In this example, as an example of the evaluation, a case of performing the evaluation of <1> the walking style in the waveform data will be described as a representative, but the evaluation of <2> the foot angle, <3> the movement of the lower half body, and <4> the movement of the upper half body are also similarly performed.

Since walking ability decreases with age, the ideal data needs to be changed in accordance with the age of the target person 211.

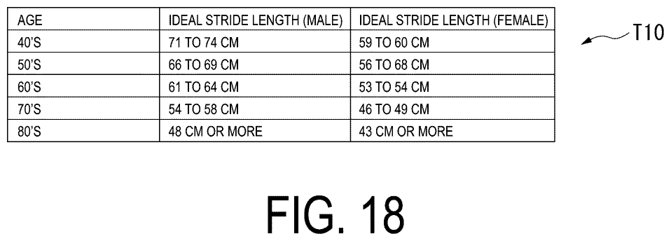

FIG. 18 is a diagram showing a tenth table T10 representing an example of age-specific ideal stride lengths according to the embodiment.

The tenth table T10 shows ideal stride lengths for men and ideal stride lengths for women for each age.

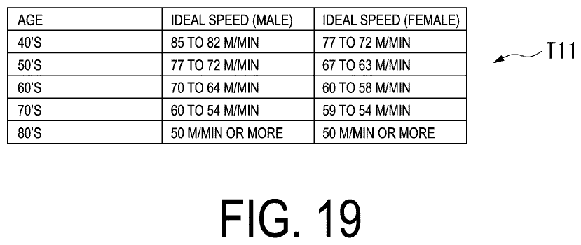

FIG. 19 is a diagram showing an eleventh table T11 representing an example of ideal walking speeds for each age according to the embodiment.

With respect to the walking speed, the eleventh table T11 shows ideal speeds for men and ideal speeds for women for each age.

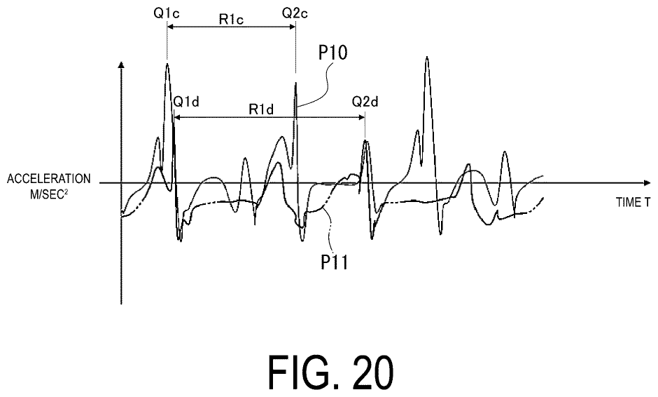

FIG. 20 is a diagram showing an example of a first walking characteristic P11 in the first pattern according to the embodiment.

In the graph shown in FIG. 20, the horizontal axis represents time [t] and the vertical axis represents acceleration [m/sec²].

The graph shows a first ideal characteristic P10 and a first walking characteristic P11 of the target person 211 in the first pattern for walking data.

In the example of FIG. 20, the first ideal characteristic P10 related to the waveform of ideal walking data is compared with the first walking characteristic P11 related to the waveform of the walking data of the target person 211 in the first pattern.

FIG. 20 shows a 1c ground contact timing Q1c and a 2c ground contact timing Q2c in the first ideal characteristic P10, a 1c walking period R1c which is one walking period corresponding to the time required for the first walking cycle, a 1d ground contact timing Q1d and a 2d ground contact timing Q2d in the first walking characteristic P11, and a 1d walking period Rid which is one walking period corresponding to the time required for the first walking cycle.

Here, in the example of FIG. 20, the characteristic related to the waveform of the walking data of the left foot is shown, but the characteristic related to the waveform of the walking data of the right foot may be used, or both of these characteristics may be used.

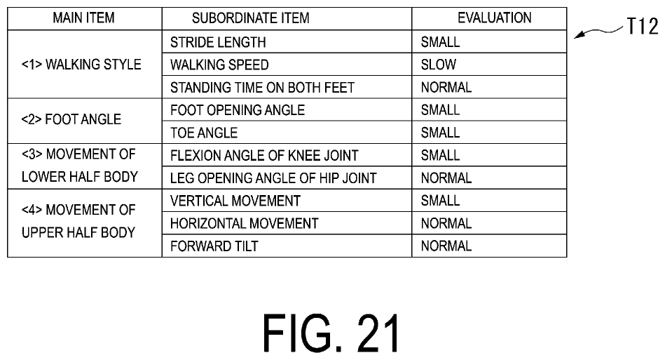

FIG. 21 is a diagram showing a twelfth table T12 representing an example of evaluation information in the first pattern according to the embodiment.

In the twelfth table T12, main items are defined for each subordinate item, and evaluations are described for each subordinate item.

The main items include <1> the walking style, <2> the foot angle, <3> the movement of the lower half body, and <4> the movement of the upper half body, which are information about the walking.

The first pattern is evaluated such that the stride length is short because amplitudes of the acceleration at the 1d ground contact timing Q1d and the 2d ground contact timing Q2d in the first walking characteristic P11 are smaller than amplitudes of the acceleration at the 1c ground contact timing Q1c and the 2c ground contact timing Q2c in the first ideal characteristic P10, and the walking speed is also slow because the 1d walking period Rid in the first walking characteristic P11 is longer than the 1c walking period R1c in the first ideal characteristic P10.

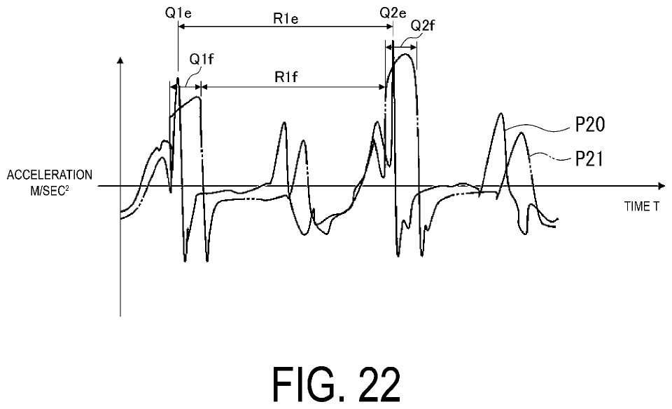

FIG. 22 is a diagram showing an example of a second walking characteristic P21 in the second pattern according to the embodiment.

In the graph shown in FIG. 22, the horizontal axis represents time [t] and the vertical axis represents acceleration [m/sec²].

With respect to the walking data, the graph shows a second ideal characteristic P20 and the second walking characteristic P21 of the target person 211 in the second pattern.

In the example of FIG. 22, the second ideal characteristic P20 related to the waveform of ideal walking data is compared with the second walking characteristic P21 related to the waveform of walking data of the target person 211 in the second pattern.

FIG. 22 shows a 1e ground contact timing Q1e and a 2e ground contact timing Q2e in the second ideal characteristic P20, a 1e walking period R1e which is one walking period corresponding to the time required for the first walking cycle, a if ground contact timing Q1f and a 2f ground contact timing Q2f in the second walking characteristic P21, and a if walking period Rif which is one walking period corresponding to the time required for the first walking cycle.

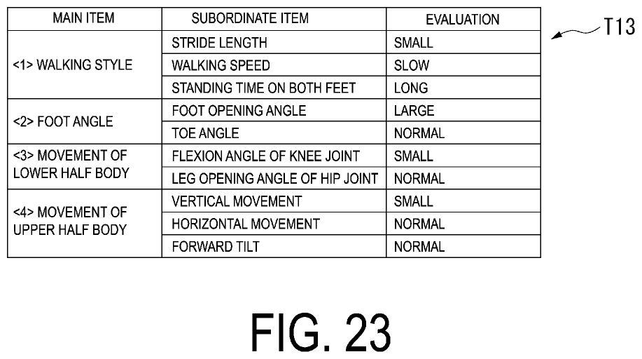

Here, in the example of FIG. 23, the characteristic related to the waveform of the walking data of the left foot is shown, but the characteristic related to the waveform of the walking data of the right foot may be used, or both of these characteristics may be used.

FIG. 23 is a diagram showing a thirteenth table T13 representing an example of evaluation information in the second pattern according to the embodiment.

In the thirteenth table T13, subordinate items are defined for each main item, and evaluations are described for each subordinate item.

The main items include <1> the walking style, <2> the foot angle, <3> the movement of the lower half body, and <4> the movement of the upper half body, which are information about the walking.

The second pattern is evaluated such that the time during which the foot is in contact with the ground is long because peak widths of the acceleration at the if ground contact timing Q1f and the 2f ground contact timing Q2f in the second walking characteristic P21 are longer than peak widths of the acceleration at the 1e ground contact timing Q1e and the 2e ground contact timing Q2e in the second ideal characteristic P20, and the walking speed is also slow because the if walking period R1f in the second walking characteristic P21 is longer than the 1e walking period R1e in the second ideal characteristic P20.

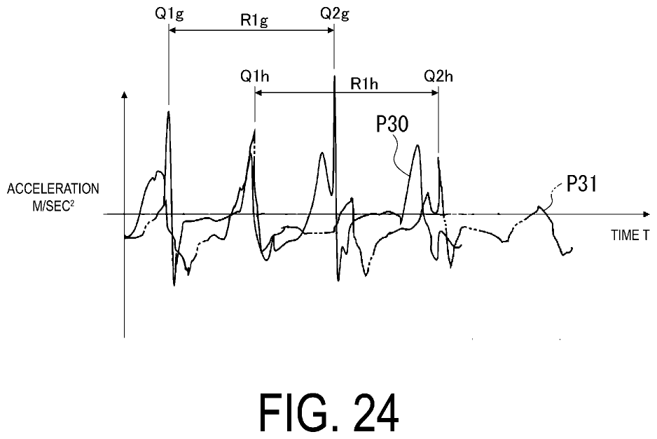

FIG. 24 is a diagram showing an example of a third walking characteristic P31 in the third pattern according to the embodiment.

In the graph shown in FIG. 24, the horizontal axis represents time [t] and the vertical axis represents acceleration [m/sec²].

With respect to the walking data, the graph shows a third ideal characteristic P30 and the third walking characteristic P31 of the target person 211 in the third pattern.

In the example of FIG. 24, the third ideal characteristic P30 related to the waveform of ideal walking data is compared with the third walking characteristic P31 related to the waveform of walking data of the target person 211 in the third pattern.

FIG. 24 shows a 1g ground contact timing Q1g and a 2g ground contact timing Q2g in the third ideal characteristic P30, a 1g walking period Rig which is one walking period corresponding to the time required for the first walking cycle, a 1h ground contact timing Qlh and a 2h ground contact timing Q2h in the third walking characteristic P31, and a 1h walking period R1h which is one walking period corresponding to the time required for the first walking cycle.

Here, in the example of FIG. 24, the characteristic related to the waveform of the walking data of the left foot is shown, but the characteristic related to the waveform of the walking data of the right foot may be used, or both of these characteristics may be used.

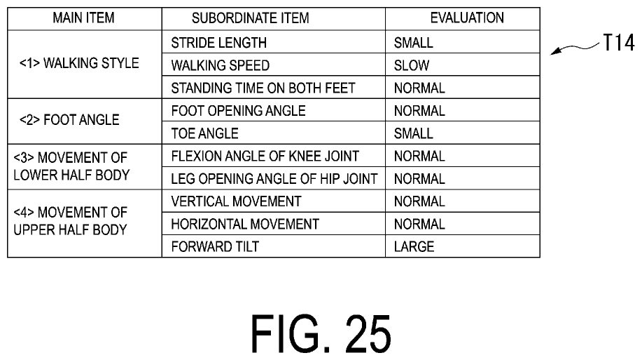

FIG. 25 is a diagram showing a fourteenth table T14 representing an example of evaluation information in the third pattern according to the embodiment.

In the fourteenth table T14, subordinate items are defined for each main item, and evaluations are described for each subordinate item.

The main items include <1> the walking style, <2> the foot angle, <3> the movement of the lower half body, and <4> the movement of the upper half body, which are information about the walking.

The third pattern is evaluated such that the stride length is short because amplitudes of the acceleration at the 1h ground contact timing Q1h and the 2h ground contact timing Q2h in the third walking characteristic P31 are smaller than amplitudes of the acceleration at the 1g ground contact timing Q1g and the 2g ground contact timing Q2g in the third ideal characteristic P30, and the walking speed is also slow because the 1h walking period R1h in the third walking characteristic P31 is longer than the 1g walking period Rig in the third ideal characteristic P30.

The instruction content will be described.

In the instruction content, it is estimated that the walking ability of the target person 211 will improve, and training and advice based on the personal information of the target person 211 are described. In addition, the training includes rehabilitation. Rehabilitation is also called rehab.

The instruction content is an instruction for improving the evaluation of the walking of the target person 211. The evaluation represents a degree of similarity to the ideal data.

The instruction content is output as individual instruction content for each target person 211 based on the target person information G1 which is personal information of the target person and the exercise evaluation information G2 which is evaluation of the walking.

Training in the instruction content will be described.

The training is classified into, for example, walking training, muscle strength training, and balance training. In addition, each category has training for different levels of difficulty.

Also, in this embodiment, categories of the walking training, the muscle strength training, and the balance training are shown, but the present disclosure is not limited thereto, and other categories may be used.

The content of each training may be changed depending on the age of the target person 211. By changing the content of the training depending on the age, for example, it is possible to reduce the risk of injury to the target person 211 and to encourage the training with a load suitable for the age.

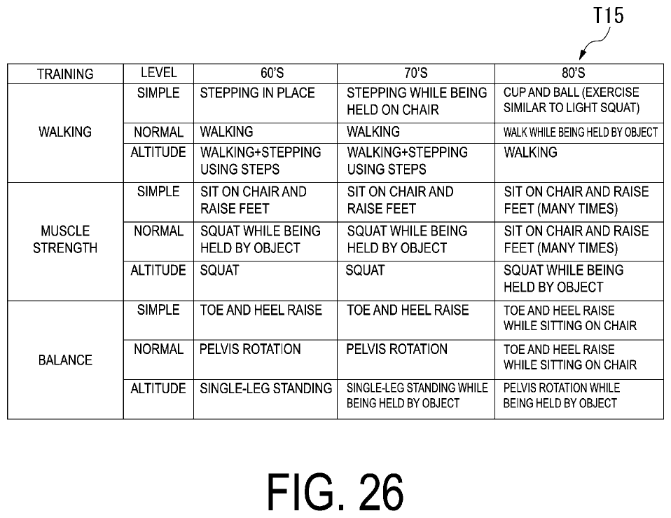

FIG. 26 is a diagram showing a fifteenth table T15 representing an example of classification of the training according to the embodiment.

In the fifteenth table T15, training contents divided by levels and classified by age groups for each category such as walking, muscle strength, or balance are defined.

Advice in the instruction content will be described.

The advice on the training which is estimated to improve the evaluation of the walking of the target person may be changed based on the target person information G1 which is the personal information of the target person.

An example of the advice in the first pattern will be described.

The first pattern is a pattern according to the example of FIGS. 10, 20, and 21.

Here, the information input to the learning model A1 as the target person information G1 may be, for example, values representing the information of each item as shown in FIG. 10, or may be other values based on the information.

Also, the information input to the learning model A1 as the exercise evaluation information G2 may be, for example, values representing the waveform data as shown in FIG. 20, values representing the evaluation results of each item as shown in FIG. 21, or other values based on one or both of them.

With respect to the first pattern, the walking training will be described.

The training content is normal level walking. The reason for selecting this is that, since the standing time on both feet is normal, she has the muscle strength for performing normal level training.

The advice content is content of suggesting walking training in a facility such as a sports gym or walking training in a park or the like. The reason for this is because it can be determined that the target person 211 lives in a city and there is a facility such as a sports gym or a park, and if the personality of the target person 211 is attracted, it can be determined that she has the personality that allows her to adapt to new things and environments.

With respect to the first pattern, the muscle strength training will be described.

The training content is to sit on a chair and raise the feet at a simple level. The reason for selecting this is that, since the toe angle is small, the muscle strength for raising the feet is weakened.

The advice content is content of not only recommending the training at home, but also suggesting the training outdoors such as on a park bench or the like. The reason for this is that, similarly to the training for the walking, it can be determined from the personality of the target person 211 that the target person 211 can cope with the suggestion of training in a new environment.

With respect to the first pattern, the balance training will be described.

The training content is normal level pelvic rotation. The reason for selecting this is that, since the horizontal movement and the forward tilt are normal, she has the muscle strength for performing normal level training using muscles for supporting her body.

The advice content is content of advising to perform the training at home. The reason for this is that the target person 211 has the personality that allows her to cope with new environments, but is not active about new environments, and thus it can be determined that training can be continued by encouraging the training in a familiar environment.

An example of advice in the second pattern will be described.

The second pattern is a pattern according to the example of FIGS. 11, 22, and 23.

Here, the information input to the learning model A1 as the target person information G1 may be, for example, values representing information of each item as shown in FIG. 11, or may be other values based on the information.

Also, the information input to the learning model A1 as the exercise evaluation information G2 may be, for example, values representing the waveform data as shown in FIG. 22, values representing the evaluation results of each item as shown in FIG. 23, or other values based on one or both of them.

With respect to the second pattern, the walking training will be described.

The training content is stepping in place at a normal level. The reason for selecting this is that, since the standing time on both feet is normal, he has the muscle strength for performing normal level training.

The advice content is a content of notifying that the results of the target person 211 are worse than the ideal data serving as a target and informing that improvement can be achieved by performing the suggested training. The reason for this is that it can be determined from the personality of the target person 211 that he has a strong tendency to want to maintain the activities of his youth, and by conveying the fact that the data is not achieved compared to the ideal data and advice to improve it, the training for enabling him to resume the activities of his youth is enthusiastically performed.

With respect to the second pattern, the muscle strength training will be described.

The training content is to sit on a chair and raise the feet at a simple level. The reason for selecting this is that the toe angle is normal, but the weight is low, and the muscle strength for raising the feet is weakened.

The advice content is content of conveying precautions to avoid injuries during training because of the risk of injury due to his physique, and conveying what kind of activities can be performed in the future by continuing the training. The reason for this is to call his attention not to be injured because the body type of the target person 211 is a low weight type.

With respect to the second pattern, the balance training will be described.

The training content is to rotate the pelvis while being held on a chair at a normal level The reason for selecting this is that, since the horizontal movement and the forward tilt are normal, he has the muscle strength for performing normal level training using muscles for supporting his body.

The advice content is content of advising to perform the training on a chair, a handrail, or the like at home. The reason for this is to call his attention not to be injured because the body type of the target person 211 is a low weight type.

An example of advice in the third pattern will be described.

The third pattern is a pattern according to the example of FIGS. 12, 24, and 25.

Here, the information input to the learning model A1 as the target person information G1 may be, for example, values representing information of each item as shown in FIG. 12, or may be other values based on the information.

Also, the information input to the learning model A1 as the exercise evaluation information G2 may be, for example, values representing the waveform data as shown in FIG. 24, values representing the evaluation results of each item as shown in FIG. 25, or other values based on one or both of them.

With respect to the third pattern, the walking training will be described.

The training content is walking at a normal level. The reason for selecting this is that, since the standing time on both feet is normal, he has the muscle strength for performing normal level training.

The advice content is content of conveying him to move by walking when he goes from home to a place for performing farm work and to be careful when he walks. The reason for this is that, since it can be determined from the personality of the target person 211 that he is reluctant to try new things, advice related to what he does on a daily basis and points to be careful about when he walks are conveyed.

With respect to the third pattern, the muscle strength training will be described.

The training content is to sit on a chair and raise the feet at a simple level. The reason for selecting this is that the toe angle is normal and the weight is usually low, but he has back pain, and thus the training is not performed according to normal level content but performed at a simple level to prevent the back pain from getting worse.

The advice content is content of giving advice regarding training postures. The reason for this is that the target person 211 has a chronic disease and performs the training in a correct posture to prevent the chronic disease from getting worse.

With respect to the third pattern, the balance training will be described.

The training content is toe raising and heel raising at a simple level. The reason for selecting this is that, since the forward tilt is large and there is a risk of falling in a front to rear direction, he performs the training so that he can hold on in the front to rear direction in the training to reduce the risk of falling.

The advice content is content of suggesting not only a training method to be performed while being held in a chair or the like at home, but also a training method to be performed while being supported by a family member. The reason for this is that the target person 211 not only performs the training by himself, but is also encouraged to continue the training by being assisted by the family member of the target person 211.

A form of the instruction report H1 will be described.

Here, any form may be used as the content described in the instruction report H1 including the instruction content or the like, or as a format of description.

For example, in the instruction report H1, one or more of sentences, figures, graphs, and the like may be described.

Learning processing will be described.

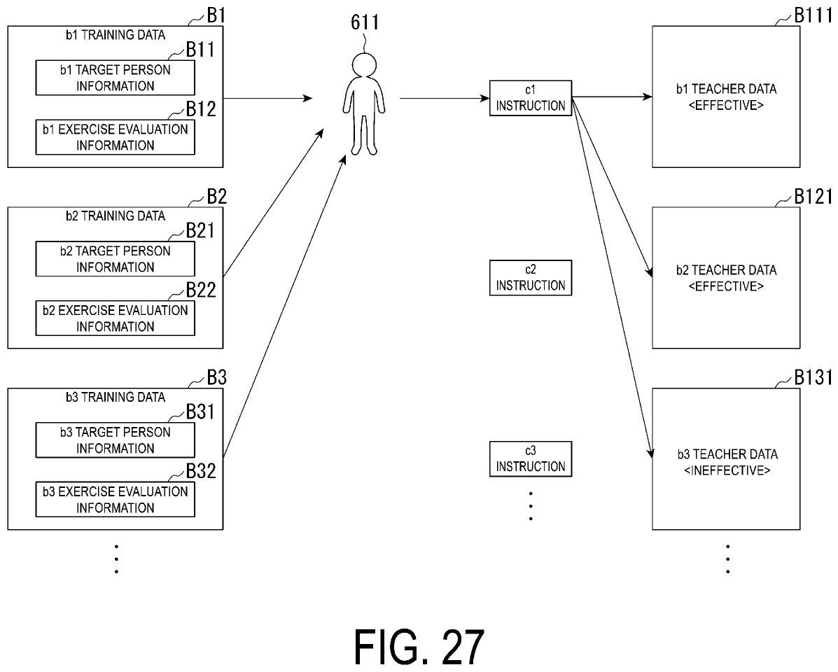

The learning processing will be described with reference to FIGS. 27 to 29.

FIG. 27 is a diagram schematically showing an example of generation of teacher data according to the embodiment. Also, training data may be called learning data or the like.

FIG. 27 shows b1 training data B1, b2 training data B2, and b3 training data B3.

The b1 training data B1 includes b1 target person information B11 and b1 exercise evaluation information B12.

The b2 training data B2 includes b2 target person information B21 and b2 exercise evaluation information B22.

The b3 training data B3 includes b3 target person information B31 and b3 exercise evaluation information B32.

Here, the b1 exercise evaluation information B12, the b2 exercise evaluation information B22, and the b3 exercise evaluation information B32 are exercise evaluation information having the same pattern B.

Also, the number of pieces of training data including exercise evaluation information having the same pattern may be, for example, four or more, or may be two, but in this embodiment, for convenience of description, a case in which the number is three will be described as an example. The number of pieces of such training data is actually, for example, a large number.

An instructor 611 may perform different kinds of instruction such as c1 instruction, c2 instruction, c3 instruction, and the like, and the c1 instruction will be described here as an example. In this embodiment, the c1 instruction is performed using an instruction report in which instruction content is described.

After the instructor 611 passes the instruction report including the content of the c1 instruction to a target person corresponding to the b1 training data B1, measurement of exercise is performed again for the target person on which the instruction has been reflected Then, the result of re-measurement after the target person receives the instruction report is used as b1 teacher data B111. The b1 teacher data B111 is, for example, exercise evaluation information obtained again for the target person and includes information about whether or not the instruction was effective.

For example, when the exercise evaluation information after the instruction is improved to such an extent that a predetermined condition is satisfied as compared with the exercise evaluation information before the instruction, the instruction is deemed effective, and in other cases, the instruction is deemed ineffective.

In this embodiment, the case in which the instruction is effective is also referred to as “there is an effect,” and a case in which the instruction is ineffective is also referred to as “there is no effect.”

Here, the case in which the b1 teacher data B111 is generated from the b1 training data B1 and the c1 instruction has been described, but similarly, b2 teacher data B121 is generated from the b2 training data B2 and the c1 instruction, and b3 teacher data B131 is generated from the b3 training data B3 and the c1 instruction.

In the example of FIG. 27, the b1 teacher data B111 and the b2 teacher data B121 are when there is an effect, and the b3 teacher data B131 is when there is no effect.

Analysis in a model being trained will be described.

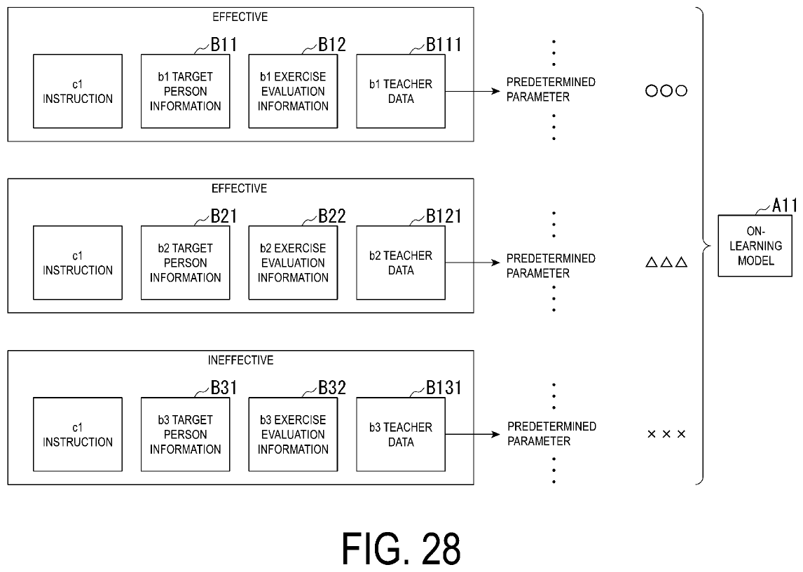

FIG. 28 is a diagram schematically showing an example of analysis in a model being trained A11 according to the embodiment.

Here, the model being trained A11 is a learning model in a state in which the learning model A1 is in a learning-in-progress stage. The learning model A1 changes from a state before learning to a trained model via the state of the model being trained A11.

From the results of the instruction, the model being trained A11 searches for a tendency as to the instruction is effective when which information is present in which parameter in the target person information.

In addition, the model being trained A11 may further determine a degree of effectiveness of the instruction. The degree of effectiveness may be represented by, for example, a numerical value representing the degree of effectiveness, and as an example, the numerical value increases as the effectiveness becomes greater.

In the example of FIG. 28, in the b1 target person information B11 of the target person corresponding to the b1 teacher data B111, the value of the predetermined parameter is “OOO.” As an example, there is one predetermined parameter, which is a “personality of a responsive type: a dependent type.”

In addition, in the b2 target person information B21 of the target person corresponding to the b2 teacher data B121, the value of the predetermined parameter is “ΔΔΔ.” As an example, there is one predetermined parameter, which is a “personality of a responsive type: a dependent type.”

Further, in the b3 target person information B31 of the target person corresponding to the b3 teacher data B131, the value of the predetermined parameter is “XXX.” As an example, there is one predetermined parameter, which is a “personality of a non-responsive type; an internal punishment type.”

The model being trained A11 analyzes the target person information to determine for which status of the target person the effect is exhibited and searches for the effective status.

In the example of FIG. 28, it is assumed that the personality of the target person corresponding to the b1 teacher data B111 and the personality of the target person corresponding to the b2 teacher data B121 represent a “responsive type: a dependent type,” but the personality of the target person corresponding to the b3 teacher data B131 is not so, but represents a “non-responsive type: an internal punishment type.”

Thus, the model being trained A11 learns that, if the c1 instruction is performed for the target person whose personality is a “responsive type: a dependent type” when the exercise evaluation information is the pattern B, there is an effect.

Also, in the example of FIG. 28, for each target person, the target person information, the exercise evaluation information before the instruction, the instruction content, and the exercise evaluation information after the instruction are used in the learning processing, but for example, for the exercise evaluation information before the instruction and the exercise evaluation information after the instruction, the information may not necessarily be used as it is, and processed information may be used as long as the pattern of the exercise evaluation information before the instruction can be identified and the effect of the instruction can be identified.

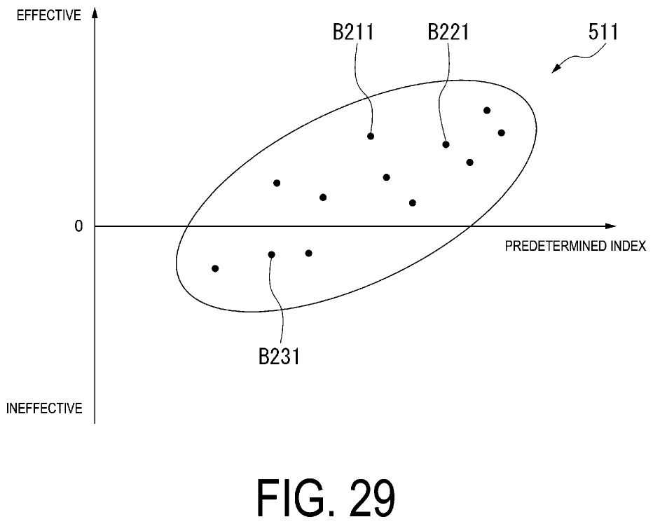

FIG. 29 is a diagram schematically showing an example of analysis content in the model being trained A11 according to the embodiment.

In the graph shown in FIG. 29, the horizontal axis represents a predetermined index, and the vertical axis represents a degree of effectiveness. On the vertical axis, there is no effect at a position of ±0, the effect increases upward in the positive range, and the reverse effect increases downward in the negative range.

FIG. 29 shows a distribution characteristic 511 according to analysis results assuming a case in which a large number of teacher data whose exercise evaluation information is the pattern B are analyzed. Also, the distribution characteristic 511 is an example for description and is not an actually obtained result.

In the example of FIG. 29, the predetermined index is, for example, a predetermined parameter itself or a value obtained from the predetermined parameter.

In the example of FIG. 29, a point corresponding to the b1 teacher data B111 corresponds to a b1 point B211, a point corresponding to the b2 teacher data B121 corresponds to a b2 point B221, and a point corresponding to the b3 teacher data B131 corresponds to a b3 point B231.

In this way, the learning model A1 learns correlations between the results of re-measurement after the target person 211 associated with the target person information G1 and the exercise evaluation information G2 receives the instruction report H1.

Also, in this embodiment, not only the information about whether or not there is an effect of the instruction but also the information about a degree of effectiveness of the instruction is used for the teacher data, but as another example, information about whether or not there is an effect of the instruction may be used for the teacher data, and information about a degree of effectiveness of the instruction may not be used.

As described above, the information processing device 1 according to this embodiment has the following configurations.

The target person information acquisition unit 111 acquires the target person information G1 including age information indicating the age of the target person 211.

The exercise evaluation information acquisition unit 112 acquires the exercise evaluation information G2 about the evaluation of the exercise of the target person 211.

The storage unit 14 stores the trained model A2. When the target person information G1 and the exercise evaluation information G2 are input, the evaluation of the exercise information G3 of the target person 211 is estimated to be improved, and the trained model A2 outputs the instruction content based on the age information.

The report output unit 113 inputs the target person information G1 and the exercise evaluation information G2 to the trained model A2 and outputs the instruction report H1 including the instruction content output from the trained model A2.

Accordingly, in the information processing device 1 according to this embodiment, it is possible to output the instruction report H1 suitable for the age of each target person 211 and support the exercise of the target person 211.

For example, the information processing device 1 can infer, for each elderly target person 211, the instruction content that improves the evaluation of the exercise of the target person 211 among a plurality of instruction contents related to the exercise including walking ability, and output the instruction report H1 including the inferred instruction content, thereby supporting improvement of the walking ability and the like of the target person 211.

For example, in the information processing device 1 according to this embodiment, the target person information G1 includes physique information about the physique of the target person 211.

When the target person information G1 and the exercise evaluation information G2 are input, the evaluation of the exercise information G3 of the target person 211 is estimated to be improved, and the storage unit 14 stores the trained model A2 that outputs the instruction content based on the age information and the physique information.

Accordingly, the information processing device 1 according to this embodiment can support the exercise based on the age and physique of the target person 211.

Also, an aspect in which the physique information of the target person 211 is not included in the target person information G1 may be used.

For example, in the information processing device 1 according to this embodiment, the target person information G1 includes personality information about the personality of the target person 211.

When the target person information G1 and the exercise evaluation information G2 are input, the evaluation of the exercise information G3 of the target person 211 is estimated to be improved, and the storage unit 14 stores the trained model A2 that outputs the instruction content based on the age information and the personality information.

accordingly, the information processing device 1 according to this embodiment can support the exercise based on the age and personality of the target person 211.

Also, an aspect in which the personality information of the target person 211 is not included in the target person information G1 may be used.

For example, in the information processing device 1 according to this embodiment, the target person information G1 includes environmental information about the place at which the target person 211 lives and the family structure of the target person 211.

When the target person information G1 and the exercise evaluation information G2 are input, the evaluation of the exercise information G3 of the target person 211 is estimated to be improved, and the storage unit 14 stores the trained model A2 that outputs the instruction content based on the age information and the environment information.

Accordingly, the information processing device 1 according to this embodiment can support the exercise based on the age and environment of the target person 211.

Also, an aspect in which the environment information of the target person 211 is not included in the target person information G1 may be used.

For example, in the information processing device 1 according to this embodiment, the exercise evaluation information G2 is information obtained by comparing the ideal exercise information for the age of the target person 211 with the exercise information G3 about the exercise of the target person 211.

Accordingly, the information processing device 1 according to this embodiment can support an exercise state of the target person 211 to approach an ideal exercise state.

Here, the ideal exercise information may be, for example, the same for all target persons 211 of the same age, or may vary for each target person 211 even if they are of the same age.

Also, an aspect in which the exercise is evaluated by a method different from comparison with the ideal exercise information may be used.

For example, the information processing device 1 according to this embodiment includes the exercise information acquisition unit 114 that acquires the exercise information G3 about the exercise of the target person 211 based on the information from the first detection device 251 attached to the target person 211.

Accordingly, the information processing device 1 according to this embodiment can acquire the exercise information G3 from the movement of the target person 211 itself.

Also, an aspect in which the function of the exercise information acquisition unit 114 that acquires the exercise information G3 based on the information from the first detection device 251 is not provided in the information processing device 1 may be used.

For example, in the information processing device 1 according to this embodiment, the first detection device 251 detects information about at least one of acceleration, position information, and angular velocity.

Accordingly, the information processing device 1 according to this embodiment can acquire the exercise information G3 based on one or more of acceleration, position information, and angular velocity from the movement of the target person 211 itself.

Also, an aspect in which exercise information based on information other than acceleration, position information, and angular velocity is acquired may be used.

For example, the information processing device 1 according to this embodiment can acquire the exercise information G3 about the exercise of the target person 211 based on the information from the second detection device 271 attached to the tool 231 for the exercise.

Accordingly, the information processing device 1 according to this embodiment can acquire the exercise information G3 from the movement of the tool 231 used for the exercise.

Also, an aspect in which the function of the exercise information acquisition unit 114 that acquires the exercise information G3 based on the information from the second detection device 271 is not provided in the information processing device 1 may be used.

For example, in the information processing device 1 according to this embodiment, the second detection device 271 detects information about at least one of acceleration, position information, and angular velocity.

Accordingly, the information processing device 1 according to this embodiment can acquire the exercise information G3 based on one or more of acceleration, position information, and angular velocity from the movement of the tool 231 used for the exercise.

Also, an aspect in which movement information based on information other than acceleration, position information, and angular velocity is acquired may be used.

For example, in the information processing device 1 according to this embodiment, the trained model A2 is a learning model that analyzes the effective parameter among the plurality of parameters of the target person information G1 during learning when the instruction content based on the target person information G1 including the plurality of parameters related to the target person 211 and the exercise evaluation information G2, and the exercise evaluation information after the instruction are input.

Accordingly, in the information processing device 1 according to this embodiment, the learning model during learning can analyze the effective parameter among the plurality of parameters of the target person information G1 based on the input information.

Also, the present disclosure is not limited thereto, and any operation for learning may be performed as an internal operation of the learning model during learning.

For example, the information processing device 1 according to this embodiment has the following configurations.

The instruction content acquisition unit 115 acquires the instruction content based on the target person information G1 including the plurality of parameters for the target person 211 and the exercise evaluation information G2 about the evaluation of the exercise of the target person 211.

The exercise information evaluation unit 116 acquires the exercise evaluation information G2 about the evaluation of the exercise of the target person 211 after the instruction.

The storage unit 14 stores the model being trained A11. When the instruction content and the exercise evaluation information after the instruction are input, the model being trained A11 analyzes the effective parameter among the plurality of parameters of the target person information G1.

Accordingly, in the information processing device 1 according to this embodiment, the model being trained A11 can analyze the effective parameter among the plurality of parameters of the target person information G1 based on the input information.

Also, in the information processing device 1 according to this embodiment, a case in which the target person 211 is a person who performs exercise for maintaining physical ability and support related to the exercise is performed, but the present disclosure is not limited thereto and may be applied to a case in which the target person 211 performs other exercise.

A program for realizing the function of any constituent unit in any device described above may be recorded on a computer-readable recording medium, and the program may be read and executed by a computer system. The “computer system” as used here is assumed to include hardware such as an operating system (OS) or peripheral devices. The “computer-readable recording medium” is a storage device such as a portable medium such as a flexible disk, a magneto-optical disk, a read only memory (ROM), a compact disc (CD)-ROM, and a hard disk built into a computer system. The “computer-readable recording medium” is assumed to include a medium that holds a program for a certain period of time, such as a volatile memory provided inside of a computer system serving as a server or a client when the program is transmitted via a network such as the Internet or a communication line such as a telephone line. The volatile memory may be a RAM. The recording medium may be a non-transitory recording medium.

The above program may be transmitted from a computer system storing the program in a storage device or the like via a transmission medium or using transmission waves in a transmission medium to another computer system. The “transmission medium” that transmits the program refers to a medium having a function of transmitting information, such as a network such as the Internet or a communication line such as a telephone line.

The above program may be for realizing some of the above-described functions. The above program may be a so-called difference file, which can realize the above-described functions in combination with a program already recorded in the computer system. The difference file may be called a difference program.

The function of any constituent unit in any device described above may be realized by a processor. Each processing in the embodiment may be realized by a processor that operates based on information such as a program and a computer-readable recording medium that stores information such as a program. In the processor, the function of each unit may be realized by individual hardware, or the function of each unit may be realized by integrated hardware. The processor may include hardware, and the hardware may include at least one of a circuit for processing digital signals and a circuit for processing analog signals. The processor may be configured using one or both of one or more circuit devices or one or more circuit elements mounted on a circuit board. For the circuit device, an integrated circuit (IC) or the like may be used, and for the circuit element, a resistor, a capacitor, or the like may be used.

The processor may be a CPU. However, the processor is not limited to the CPU, and various processors such as a graphics processing unit (GPU) or a digital signal processor (DSP) may be used. The processor may be a hardware circuit using an application-specific integrated circuit (ASIC). The processor may be configured by a plurality of CPUs or may be configured by a hardware circuit including a plurality of ASICs. The processor may be configured by a combination of a plurality of CPUs and a hardware circuit including a plurality of ASICs. The processor may include one or more of an amplifier circuit, a filter circuit, and the like for processing analog signals.

Although the embodiment has been described in detail with reference to the drawings, specific configurations are not limited to this embodiment and include designs and the like without departing from the gist of the present disclosure.

### APPENDIX

### <Configuration Example 1> to <Configuration Example 13> Will be Shown

### Configuration Example 1

An information processing device including:
a target person information acquisition unit configured to acquire target person information including age information indicating an age of a target person;
an exercise evaluation information acquisition unit configured to acquire exercise evaluation information about evaluation of an exercise of the target person;
a storage unit configured to store a trained model in which, when the target person information and the exercise evaluation information are input, evaluation of exercise information of the target person is estimated to be improved, and instruction content based on the age information is output; and
a report output unit configured to input the target person information and the exercise evaluation information to the trained model and output an instruction report including the instruction content output from the trained model.

### Configuration Example 2

The information processing device according to <Configuration Example 1>, wherein
the target person information includes physique information about a physique of the target person, and
the storage unit stores the trained model in which, when the target person information and the exercise evaluation information are input, the evaluation of the exercise information of the target person is estimated to be improved, and instruction content based on the age information and the physique information is output.

### Configuration Example 3

The information processing device according to <Configuration Example 1> or <Configuration Example 2>, wherein
the target person information includes personality information about a personality of the target person, and
the storage unit stores the trained model in which, when the target person information and the exercise evaluation information are input, the evaluation of the exercise information of the target person is estimated to be improved, and instruction content based on the age information and the personality information is output.

### Configuration Example 4

The information processing device according to any one of <Configuration Example 1> to <Configuration Example 3>, wherein
the target person information includes environmental information about a place at which the target person lives and a family structure of the target person, and
the storage unit stores the trained model in which, when the target person information and the exercise evaluation information are input, the evaluation of the exercise information of the target person is estimated to be improved, and instruction content based on the age information and the environment information is output.

### Configuration Example 5

The information processing device according to any one of <Configuration Example 1> to <Configuration Example 4>, wherein
the exercise evaluation information is information obtained by comparing ideal exercise information for the age of the target person with exercise information about the exercise of the target person.

### Configuration Example 6

The information processing device according to any one of <Configuration Example 1> to <Configuration Example 5>, further including an exercise information acquisition unit configured to acquire the exercise information about the exercise of the target person based on information from a first detection device attached to the target person.

### Configuration Example 7

The information processing device according to <Configuration Example 6>, wherein
the first detection device detects information about at least one of acceleration, position information, and angular velocity.

### Configuration Example 8

The information processing device according to any one of <Configuration Example 1> to <Configuration Example 7>, wherein
the trained model is a learning model configured to analyze an effective parameter among a plurality of parameters of the target person information during learning when the instruction content based on the target person information including the plurality of parameters related to the target person and the exercise evaluation information and the exercise evaluation information after the instruction are input.

It is also possible to provide an information processing method performed by the information processing device described above.

### Configuration Example 9

An information processing method including:
acquiring target person information including age information indicating an age of a target person using a target person information acquisition unit of an information processing device;
acquiring exercise evaluation information about evaluation of an exercise of the target person using an exercise evaluation information acquisition unit of the information processing device;
storing, using a storage unit of the information processing device, a trained model in which, when the target person information and the exercise evaluation information are input, evaluation of exercise information of the target person is estimated to be improved, and instruction content based on the age information is output; and
inputting the target person information and the exercise evaluation information to the trained model and outputting an instruction report including the instruction content output from the trained model using a report output unit of the information processing device.

It is also possible to provide a program recording medium that records a program executed by the processor in the information processing device as described above.

### Configuration Example 10

A program recording medium configured to record a program, the program causing a computer to execute:
a target person information acquisition function configured to acquire target person information including age information indicating an age of a target person;
an exercise evaluation information acquisition function configured to acquire exercise evaluation information about evaluation of an exercise of the target person; and
a report output function configured to input the target person information and the exercise evaluation information to a trained model stored in a storage unit configured to store the trained model in which, when the target person information and the exercise evaluation information are input, evaluation of exercise information of the target person is estimated to be improved, and instruction content based on the age information is output, and output an instruction report including the instruction content output from the trained model.

### Configuration Example 11

An information processing device including:
an instruction content acquisition unit configured to acquire instruction content based on target person information including a plurality of parameters related to a target person and exercise evaluation information about evaluation of an exercise of the target person;
an exercise information evaluation unit configured to acquire the exercise evaluation information about the evaluation of the exercise of the target person after the instruction; and
a storage unit configured to store a model being trained in which, when the instruction content and the exercise evaluation information after the instruction are input, an effective parameter among the plurality of parameters of the target person information is analyzed.

It is also possible to provide a model being trained as described above.

### Configuration Example 12

A model being trained, wherein, when instruction content based on target person information including a plurality of parameters related to a target person and exercise evaluation information about evaluation of an exercise of the target person, and the exercise evaluation information after the instruction are input, an effective parameter among the plurality of parameters of the target person information is analyzed.

It is also possible to provide an information processing method performed by the information processing device as described above.

### Configuration Example 13

An information processing method including:
acquiring instruction content based on target person information including a plurality of parameters related to a target person and exercise evaluation information about evaluation of an exercise of the target person using an instruction content acquisition unit of an information processing device;
acquiring the exercise evaluation information about the evaluation of the exercise of the target person after the instruction using an exercise information evaluation unit of the information processing device; and
storing, using a storage unit of the information processing device, a model being trained in which, when the instruction content and the exercise evaluation information after the instruction are input, an effective parameter among the plurality of parameters of the target person information is analyzed.

1. An information processing device comprising:
a target person information acquisition unit configured to acquire target person information including age information indicating an age of a target person;
an exercise evaluation information acquisition unit configured to acquire exercise evaluation information about evaluation of an exercise of the target person;
a storage unit configured to store a trained model in which, when the target person information and the exercise evaluation information are input, evaluation of exercise information of the target person is estimated to be improved, and instruction content based on the age information is output; and
a report output unit configured to input the target person information and the exercise evaluation information to the trained model and output an instruction report including the instruction content output from the trained model.

2. The information processing device according to claim 1, wherein
the target person information includes physique information about a physique of the target person, and
the storage unit stores the trained model in which, when the target person information and the exercise evaluation information are input, the evaluation of the exercise information of the target person is estimated to be improved, and instruction content based on the age information and the physique information is output.

3. The information processing device according to claim 1, wherein
the target person information includes personality information about a personality of the target person, and
the storage unit stores the trained model in which, when the target person information and the exercise evaluation information are input, the evaluation of the exercise information of the target person is estimated to be improved, and instruction content based on the age information and the personality information is output.

4. The information processing device according to claim 1, wherein
the target person information includes environmental information about a place at which the target person lives and a family structure of the target person, and
the storage unit stores the trained model in which, when the target person information and the exercise evaluation information are input, the evaluation of the exercise information of the target person is estimated to be improved, and instruction content based on the age information and the environment information is output.

5. The information processing device according to claim 1, wherein
the exercise evaluation information is information obtained by comparing ideal exercise information for the age of the target person with exercise information about the exercise of the target person.

6. The information processing device according to claim 1, further comprising an exercise information acquisition unit configured to acquire the exercise information about the exercise of the target person based on information from a first detection device attached to the target person.

7. The information processing device according to claim 6, wherein
the first detection device detects information about at least one of acceleration, position information, and angular velocity.

8. The information processing device according to claim 1, wherein
the trained model is a learning model configured to analyze an effective parameter among a plurality of parameters of the target person information during learning when the instruction content based on the target person information including the plurality of parameters related to the target person and the exercise evaluation information and the exercise evaluation information after the instruction are input.

9. An information processing method comprising:
acquiring target person information including age information indicating an age of a target person using a target person information acquisition unit of an information processing device;
acquiring exercise evaluation information about evaluation of an exercise of the target person using an exercise evaluation information acquisition unit of the information processing device;
storing, using a storage unit of the information processing device, a trained model in which, when the target person information and the exercise evaluation information are input, evaluation of exercise information of the target person is estimated to be improved, and instruction content based on the age information is output; and
inputting the target person information and the exercise evaluation information to the trained model and outputting an instruction report including the instruction content output from the trained model using a report output unit of the information processing device.

10. (canceled)

11. (canceled)

12. A model being trained, wherein, when instruction content based on target person information including a plurality of parameters related to a target person and exercise evaluation information about evaluation of an exercise of the target person, and the exercise evaluation information after the instruction are input, an effective parameter among the plurality of parameters of the target person information is analyzed.

13. (canceled)
